(19) United States
(12) Patent Application Publication
(10) Pub. No.: US 2023/0338719 A1
(43) Pub. Date: Oct. 26, 2023

(54) SUBSTANCE DELIVERING CAPSULE

(71) Applicant: Novo Nordisk A/S, Bagsvaerd (DK)

(72) Inventors: Adam Jun Bohr, Frederiksberg (DK); Mikkel Oliver Bruun-Jespersen, Copenhagen N (DK); Jorrit Jeroen Water, Frederiksberg (DK); Brian Mouridsen, Fredensborg (DK); Bozhidar Nikolaev Kozhuharov, Soeborg (DK)

(21) Appl. No.: 18/034,575

(22) PCT Filed: Nov. 1, 2021
(86) PCT No.: PCT/EP2021/080288

§ 371 Date: Apr. 28, 2023

(30) Foreign Application Priority Data
Nov. 2, 2020 (EP) 20205308.8

(51) Int. Cl. A61M 31/00; A61K 9/48

(52) U.S. Cl. CPC A61M 31/002; A61K 9/4808

(57) ABSTRACT

1. A capsule device (100) suitable for swallowing into a lumen of a gastrointestinal tract of a patient and for intestinal delivery of a therapeutic substance from the capsule device, wherein the capsule device (100) comprises: a) a housing portion (110, 115, 120), b) an activation element (130) movable relative to the housing portion from an expanded pre-delivery configuration into a non-expanded delivery configuration, c) a therapeutic substance comprised in a reservoir (A), and d) a delivery assembly comprising an actuator (140) comprising a potential energy source and a delivery outlet (190). The activation element (130) is movable by intestinal wall peristaltic contraction from the ex-panded pre-delivery configuration into the non-expanded delivery configuration to cause the actuator (140) to deliver the therapeutic substance through the delivery outlet (190).

The present invention relates to drug delivery devices suitable for ingestion into a lumen of the gastrointestinal tract for delivery of a drug substance to a subject user.

### BACKGROUND OF THE INVENTION

In the disclosure of the present invention reference is mostly made to the treatment of diabetes by delivery of insulin, however, this is only an exemplary use of the present invention.

May people suffer from diseases, such as diabetes, which requires them to receive injections of drugs on a regular and often daily basis. To treat their disease these people are required to perform different tasks which may be considered complicated and may be experienced as uncomfortable. Furthermore, it requires them to bring injection devices, needles and drugs with them when they leave home. It would therefore be considered a significant improvement of the treatment of such diseases if treatment could be based on oral intake of tablets or capsules.

However, such solutions are very difficult to realise since protein-based drugs will be degraded and digested rather than absorbed when ingested.

To provide a working solution for delivering insulin into the bloodstream through oral intake, the drug has to be delivered firstly into a lumen of the gastrointestinal tract and further into the wall of the gastrointestinal tract (lumen wall). This presents several challenges among which are: (1) The drug has to be protected from degradation or digestion by the acid in the stomach. (2) The drug has to be released while being in the stomach, or in the lower gastrointestinal tract, i.e. after the stomach, which limits the window of opportunity for drug release. (3) The drug has to be delivered at the lumen wall to limit the time exposed to the degrading environ-ment of the fluids in the stomach and in the lower gastrointestinal tract. If not released at the wall, the drug may be degraded during its travel from point of release to the wall or may pass through the lower gastrointestinal tract without being absorbed, unless being protected against the decomposing fluids.

Capsule devices have been proposed for delivery of a drug substance into a lumen or lumen wall. After insertion of the capsule, such as by swallowing the capsule into the GI system of the subject, drug delivery may be performed using an actuator which forces the drug substance from a reservoir through an outlet. Typical capsule devices include a drug reservoir comprising a slidable piston arranged between the actuator, such as a compressed spring or a gas expansion unit, and the liquid drug substance in the reservoir.

Triggering of delivery for an orally delivered capsule device has been the subject of much attention. In US 2020/171287 a system is suggested wherein a separation valve opens up in response to compressive forces applied by a peristaltic contraction within the small intestine in order to control inflation of a balloon.

For swallowable capsule devices configured for delivery of a therapeutic substance in the intestinal tract it is generally a challenge to ensure a consistent performance of delivery and drug substance deposition in the tissue of the lumen wall irrespective of the varying conditions in the gastrointestinal system.

Having regard to the above, it is an object of the present invention to provide a capsule device which is improved relative to prior art capsule devices.

### DISCLOSURE OF THE INVENTION

In the disclosure of the present invention, embodiments and aspects will be described which will address one or more of the above objects or which will address objects apparent from the below disclosure as well as from the description of exemplary embodiments.

Recently, there has been numerous attempts to engineer devices that can deliver drugs specifically via the small intestine, but it has proven challenging to ensure proximity to the intestinal wall and activation at the right time. It is essential that such devices have a mechanism for achieving intestinal proximity that is independent of variations in intestinal diameter, morphol-ogy and food conditions.

Thus, in a first aspect of the invention, a capsule device is provided which is suitable for swallowing into a lumen of a gastrointestinal tract of a patient and for intestinal delivery of a therapeutic substance from the capsule device, wherein the capsule device comprises:
a housing portion,
an activation element movable relative to the housing portion from an expanded pre-delivery configuration into a non-expanded delivery configuration, the activation element being configured for intestinal wall interaction so that the activation element is moved, due to intestinal wall peristaltic contraction, from the expanded pre-delivery configuration into the non-ex-panded delivery configuration,
a therapeutic substance, and
a delivery assembly comprising a delivery outlet, wherein the delivery assembly is configured for deployment responsive to operation of the activation element to deliver the therapeutic substance through the delivery outlet,

wherein the delivery assembly comprises an actuator comprising a potential energy source, wherein the actuator is configured for driving delivery of the therapeutic substance through the delivery outlet upon release of energy from the potential energy source, and wherein the activation element is configured to initiate delivery of the therapeutic substance as the activation element moves from the expanded pre-delivery configuration into the non-expanded de-livery configuration.

The capsule according to the first aspect offers improved and consistent proximity or contact between the delivery outlet and the tissue of a lumen wall in the gastrointestinal system, and wherein initialization of substance delivery is optimally synchronized with contractions occurring due to peristaltic motion of the intestines at the delivery site. Due to the potential energy being stored in the potential energy source, and thus being available from the onset of a peristaltic contraction, the delivery of the therapeutic substance from the capsule device will become optimally synchronized with the onset of a peristaltic contraction. This serves to ensure correct tissue proximity during the entire duration of drug delivery.

The therapeutic substance may be stored in a reservoir (A) arranged within the capsule device, such as within the housing of the capsule device. Typically, the therapeutic substance will be available in reservoir (A) when the capsule device is handed out to the user or, alternatively, the substance will be filled into the capsule only prior to swallowing.

In some forms, when swallowing the device, the reservoir (A) accommodates an amount of the therapeutic substance and wherein, in use, and prior to the activation element moves from the expanded pre-delivery configuration into the non-expanded delivery configuration for initializing delivery, the potential energy source holds an amount of stored potential energy sufficient to deliver all of said amount of therapeutic substance through the delivery outlet. In other forms, the potential energy source holds an amount of stored potential energy sufficient to deliver at least 50 percent, such as at least 60 percent, such as at least 70 percent, such as at least 80 percent, such as at least 90 percent and such as at least 95%, of said amount of therapeutic substance through the delivery outlet.

In some forms, the potential energy stored in the energy source is depleted as the therapeutic substance is delivered from reservoir (A) through the drug outlet.

In some variants, the activation element is operably connected to the actuator for actuating release of energy, e.g. stored potential energy comprised within the potential energy source, from the actuator upon movement of the activation element from the expanded pre-delivery configuration to the non-expanded delivery configuration to thereby initiate delivery of the therapeutic substance.

In some embodiments, the capsule device comprises a delivery gate being arranged to control transport of the therapeutic substance from the reservoir through the delivery outlet, wherein the activation element is operably connected to the delivery gate for initializing delivery upon movement of the activation element from the expanded pre-delivery configuration to the non-expanded delivery configuration.

In some embodiments, the activation element mechanically interconnects with the delivery gate so that movement of the activation element from the expanded pre-delivery configuration to the non-expanded delivery configuration operates the delivery gate from a non-delivery state to a delivery state. The activation element may in some embodiments be connected to the delivery gate by means of a mechanical transmission so that movement of the activation element mechanically forces the delivery gate to initialize delivery.

In some forms the mechanical transmission is provided in a manner so that a peristaltic contraction force of the small intestine is transmitted via the activation element and via the mechanical transmission onto the delivery gate to mechanically open up the delivery gate to initialize delivery through the drug outlet.

In some embodiments, the activation element is releasably maintained in a non-expanded pre-delivery configuration and configured for being released for movement relative to the housing portion from the non-expanded pre-delivery configuration to the expanded pre-delivery configuration.

The capsule may be provided so that the activation element is releasably maintained either in the non-expanded pre-delivery configuration or in the expanded pre-delivery configuration by a releasable retainer. For example, the capsule may be provided so that the activation element is releasably maintained in the non-expanded pre-delivery configuration by a degradable or dissolvable retainer. The degradable or dissolvable retainer may be configured to dissolve or degrade when subjected to a biological fluid upon sufficient time of exposure to thereby release the activation element for movement into the expanded pre-delivery configuration. Alternatively, the degradable or dissolvable retainer may be configured to dissolve or degrade upon a change in at least one chemical property of the gastric fluid that surrounds the exterior of the capsule device during its passage through the gastrointestinal tract of the user/patient.

In different embodiments the releasable retainer is provided as an environmentally-sensitive mechanism such as a GI tract environmentally-sensitive mechanism. The GI tract environmen-tally-sensitive mechanism wherein the releasable retainer is characterised by at least one of the group comprising:
a) the releasable retainer comprises a material that degrades, erodes and/or dissolves due to a change in pH in the GI tract;
b) the releasable retainer comprises a material that degrades, erodes and/or dissolves due to a pH in the GI tract;
c) the releasable retainer comprises a material that degrades, erodes and/or dissolves due to a presence of an enzyme in the GI tract; and
d) the releasable retainer comprises a material that degrades, erodes and/or dissolves due to a change in concentration of an enzyme in the GI tract.

In some embodiments an activation element biasing unit is comprised by the capsule device and configured to urge the activation element for movement from the non-expanded pre-delivery configuration to the expanded pre-delivery configuration. The activation element biasing unit may in different variants comprise one or more of a swelling material portion, a spring, a compressed gas, and a gas generator. In some forms, the potential energy stored in the actuator may provide a force that urges the activation element towards movement from the non-expanded pre-delivery configuration to the expanded pre-delivery configuration.

In different embodiments of the actuator the potential energy source may be provided in the form of one or more of a pre-loaded spring, a compressed gas storage unit, and a gas generating expansion unit.

The actuator may be arranged within, or in association with, an actuation chamber (B) defined by the capsule device. A movable separator may be arranged between the actuation chamber (B) and the reservoir (A). The movable separator may in some embodiments be provided as or comprise a piston which is arranged slidable in the reservoir (A). In other forms the movable separator comprises a flexible membrane which is separating the drive force, e.g., a pressurized gas in the actuation chamber (B), and the therapeutic substance accommodated in the reservoir (A). In some forms the flexible membrane may be provided as a bag or similar enclosure having a single opening at the drug outlet for fluid communication through the drug outlet.

In embodiments wherein the actuator comprises a compressed gas storage unit, a pressurized gas canister may be provided gas enclosure with a puncturable seal that seals towards the actuation chamber. In such embodiment, the activation element connects via a mechanical transmission to a puncturing element so that movement of the activation element from the expanded pre-delivery configuration into the non-expanded delivery configuration causes relative movement between the puncturing element and the puncturable seal so as to puncture the seal. This allows the gas of the pressurized gas canister to flow unhindered to the actuation chamber (B) and drive out therapeutic substance from the drug outlet. The pressurized gas canister may comprise an amount of pressurized gas sufficient to drive out the entire expellable amount of therapeutic substance from reservoir (A).

The potential energy source may in other embodiments be provided so that it comprises a gas generating expansion unit that, by means of a gas generator, is configured for generating gas at elevated gas pressure in the actuation chamber (B).

In some embodiments the gas generator associated with the actuation chamber (B) comprises effervescent material and wherein the capsule housing portion comprises a fluid inlet portion leading to the effervescent material.

In some forms of the capsule device, the fluid inlet portion initially comprises an enteric coating adapted to dissolve when subjected to a biological fluid within the lumen, wherein biological fluid within the lumen is allowed to flow through the fluid inlet portion upon dissolving of the enteric coating to cause contact between the biological fluid and the effervescent material.

In further embodiments the fluid inlet portion comprises a semi-permeable membrane allowing biological fluid within the lumen to migrate through the semi-permeable membrane and enter into contact with the effervescent material.

In alternative embodiments, the capsule device comprises a liquid compartment filled with a liquid, wherein the gas generator comprises an effervescent material configured to generate gas when subjected to contact with liquid from the liquid compartment, and wherein upon release of the releasable retainer enables contact between the effervescent material and the liquid.

In still other embodiments, the gas generator comprises at least a first reactant and a second reactant configured to generate gas in the actuation chamber (B) upon contact between the first reactant and the second reactant.

In some embodiments the capsule device further comprises a pressure operated latch arrangement for controlling operation of the delivery gate, the pressure operated latch arrangement being configured to prevent the delivery gate to be operated for initializing delivery, but, upon generation of elevated gas pressure above a predefined level in the actuation chamber (B), and/or upon generation of elevated liquid pressure above a predefined level in the reservoir (A), enable operation of the delivery gate to initialize delivery.

In some forms the pressure operated latch arrangement is configured to block the activation element from moving from the non-expanded pre-delivery configuration to the expanded pre-delivery configuration, and to release upon elevated gas pressure being generated above a predefined level in the actuation chamber (B), and/or elevated liquid pressure being generated above a predefined level in the reservoir (A), to enable operation of the activation element from the non-expanded pre-delivery configuration to the expanded pre-delivery configuration.

In other forms the pressure operated latch arrangement is configured to block the activation element from moving from the expanded pre-delivery configuration to the non-expanded de-livery configuration, and to release upon elevated gas pressure being generated above a pre-defined level in the actuation chamber (B), and/or elevated liquid pressure being generated above a predefined level in the reservoir (A), to enable operation of the activation element from the expanded pre-delivery configuration to the non-expanded delivery configuration.

The delivery gate may be provided so that it defines a first gate element and a second gate element associated with, or formed by, the housing portion and the activation element, respectively, and wherein the first gate element and the second gate element define cooperating elements of an openable seal or openable valve associated with the delivery outlet.

In some embodiments said openable seal or openable valve associated with the delivery outlet is operable between a non-openable state and an openable state, and wherein capsule device is so adapted that the openable seal or openable valve is configured to move from the non-openable state into the openable state subsequent to movement of the activation element from the non-expanded pre-delivery configuration to the expanded pre-delivery configuration. The openable seal or openable valve may be configured to move from the non-openable state into the openable state upon elevated gas pressure above a predefined level in the actuation chamber (B), and/or elevated liquid pressure above a predefined level in the reservoir (A), being generated.

The openable seal or openable valve may comprise a bi-stable diaphragm configured to move from the non-openable state into the openable state upon elevated liquid pressure above a predefined level in the reservoir (A) being generated.

In some embodiments, the lumen, such as the small intestines, defines a lumen wall, wherein the drug outlet comprises a jet nozzle arrangement configured for needleless jet delivery. In this way, the ingestible capsule device does not include sharp needle points and a mechanism which actuates and retracts the needle is also not required.

the capsule device may in different embodiments be configured to act by one of liquid jet de-livery of a liquid formulation, jet delivery of a powder formulation, needle delivery of a liquid formulation, and solid dose delivery of a solid dose formulation.

Existing jet injector systems for jet delivery are known in the art. A skilled person would un-derstand how to select an appropriate jet injector that provides the correct jetting power to deliver the therapeutic substance into the lumen wall, for example from WO 2020/106,750 (PROGENITY INC). Further details and examples are provided further on in the application.

For needle-less jet injection embodiments, the capsule may be configured to expel drug substance through the nozzle arrangement with a penetration velocity allowing the drug substance to penetrate tissue of the lumen wall.

In other forms of the capsule, the drug outlet comprises an injection needle wherein the drug substance is expellable through the injection needle. The injection needle associated with the drug outlet may be configured for movement between a non-exposed state and an exposed state for penetrating intestinal tissue by the injection needle upon movement of the activation element from the expanded pre-delivery configuration into the non-expanded delivery configuration.

In certain embodiments, the activation element is further operable from the non-expanded de-livery configuration into an expanded post-delivery configuration, wherein the injection needle moves from the exposed state to the non-exposed state upon movement of the activation element from the non-expanded delivery configuration into the expanded post-delivery configuration. Hence, upon consecutive peristaltic contractions, the needle initially arranged in a first non-exposed state within the capsule device is brought into the exposed state to penetrate tissue, and subsequently, after delivery, be brought into a second non-exposed state within the capsule device for safe disposal.

In exemplary embodiments, the capsule device is configured for swallowing by a patient and travelling into a lumen of a gastrointestinal tract of a patient for delivery at a target location, such as the small intestines or the large intestines. The capsule of the device may be shaped and sized to allow it to be swallowed by a subject, such as a human.

In some forms the capsule defines an elongated object that extends along a longitudinal axis, and wherein the activation element forms an element that is radially movable by swivelling or linear movement relative to the housing portion. Non-limiting embodiments include capsule devices wherein the activation element is provided as a rigid member, such as forming a wing, a leg or an arm, that connects to the housing portion via a pivot joint or a linear guide.

In embodiments wherein the housing portion comprises an elongated wall section extending along the longitudinal axis, said drug outlet may, to facilitate tissue interaction, be arranged to point laterally outwards with respect to elongated wall section. In some embodiments the drug outlet may be fixedly mounted relative to the housing portion. In other embodiments, the drug outlet may be fixedly mounted relative to the activation element. In embodiments, wherein a portion of the activation element moves in an activation direction relative to the housing when moving from the expanded pre-delivery configuration to the non-expanded delivery configuration, the drug outlet may be oriented to point in a direction parallel with the activation direction, either in the activation direction or in a direction opposite to the activation direction.

In further forms of the capsule device, the drug outlet forms a drug outlet assembly which comprises a multitude of separate drug outlets.

By the above arrangements an orally administered therapeutic substance can be delivered safely and reliably into the intestinal wall of a living mammal subject. Other advantageous solutions are provided by each the embodiments described in the appended description.

As used herein, the terms “drug”, “drug substance”, “drug product” or “therapeutic substance” is meant to encompass any drug formulation or beneficial agent capable of being delivered into or onto the specified target site. The drug may be a single drug compound, a premixed or co-formulated multiple drug compound, or even a drug product being mixed by two or more separate drug constituents wherein the mixing is performed either before or during expelling. Representative drugs include pharmaceuticals such as peptides (e.g. insulins, insulin containing drugs, GLP-1 containing drugs as well as derivatives thereof), proteins, and hormones, biologically derived or active agents, hormonal and gene-based agents, nutritional formulas and other substances in both solid, powder or liquid form. Specifically, the drug may be an insulin or a GLP-1 containing drug, this including analogues thereof as well as combinations with one or more other drugs.

### BRIEF DESCRIPTION OF THE DRAWINGS

In the following embodiments of the invention will be described with reference to the drawings, wherein

FIG. 1 is a cross-sectional side view of an ingestible capsule 100 according to a first embodiment of the invention,

FIG. 2 is an exploded perspective view of components of capsule 100 according to the first embodiment,

FIGS. 3a-3c are perspective views of a needle hub, needle and latch mechanism of capsule 100 according to the first embodiment, each view showing the arrangement depicted in one of three consecutive states,

FIGS. 4a-4f are perspective partially cut views of capsule 100 according to the first embodiment, each view showing the capsule 100 depicted in one of six consecutive states,

FIGS. 5a-5c are cross-sectional side views of a capsule 200 according to a second embodiment, each view showing the capsule 200 depicted in one of three consecutive states,

FIG. 6 is a cross-sectional side view of a capsule 300 according to a third embodiment,

FIG. 7a is a partly cut, cross-sectional side view of a capsule 400 according to a fourth embodiment, the view showing the capsule in a state prior to ingestion,

FIGS. 7b-7d show cross-sectional side and cross-sectional end views of capsule 400 according to the fourth embodiment, each view showing the capsule 400 depicted in one of three consecutive states following ingestion,

FIGS. 8a-8c are cross-sectional side views of a capsule 500 according to a fifth embodiment, each view showing the capsule 500 depicted in one of three consecutive states, and

FIGS. 9a-9c are schematic drawings illustrating the operating principle of an example embodiment of a pressure operated latch arrangement.

In the figures like structures are mainly identified by like reference numerals.

### DESCRIPTION OF EXEMPLARY EMBODIMENTS

When in the following terms such as “up” and “down”, “upper” and “lower”, “right” and “left”, “horizontal” and “vertical” or similar relative expressions are used, these only refer to the appended figures and not necessarily to an actual situation of use. The shown figures are schematic representations for which reason the configuration of the different structures as well as their relative dimensions are intended to serve illustrative purposes only. When the term member or element is used for a given component it generally indicates that in the described embodiment the component is a unitary component, however, the same member or element may alternatively comprise a number of sub-components just as two or more of the described components could be provided as unitary components, e.g. manufactured as a single injection moulded part. The terms “assembly” and “subassembly” do not imply that the described components necessarily can be assembled to provide a unitary or functional assembly or subassembly during a given assembly procedure but is merely used to describe components grouped together as being functionally more closely related.

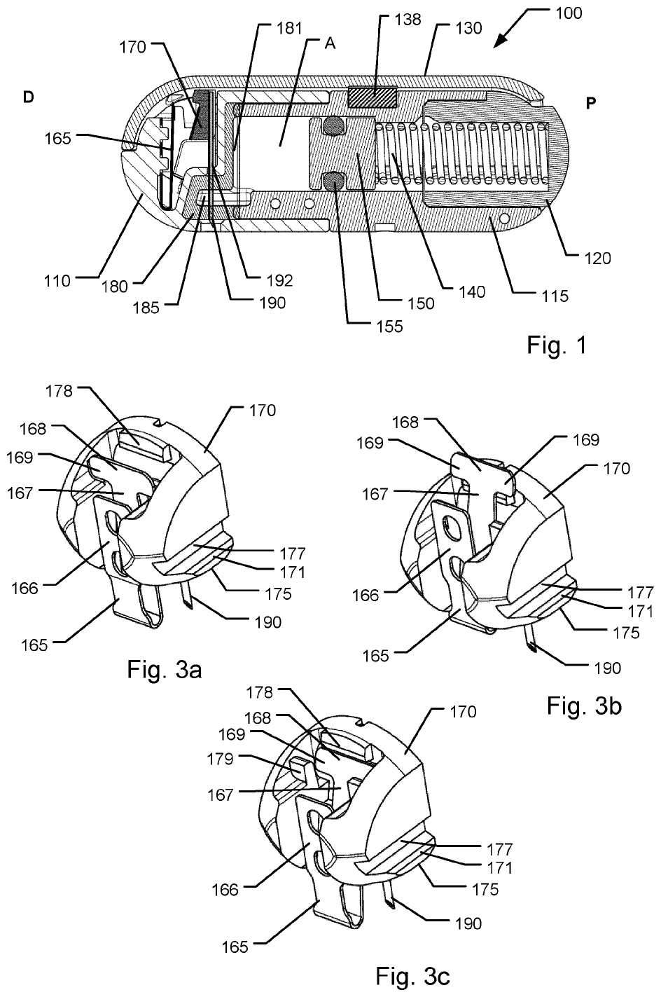

With reference to FIGS. 1 through 4f, a first embodiment of a drug delivery device in accordance with the invention will be described, the embodiment being designed to provide a capsule device 100 sized and shaped to be ingested by a subject, such as a patient, the device being configured for subsequently deploying a triggerable expelling system incorporated in the capsule device that, when triggered in a target lumen of the patient, causes a dose of a liquid drug to be expelled through a drug outlet provided at an external portion of the capsule device 100. It is to be noted that the disclosed ingestible capsule device 100, in the following referred to simply as “capsule”, is only exemplary and, in accordance with the invention, may be provided in other forms having different capsule outer shapes. Also, although the shown outlet comprises a single injection needle configured to be inserted into mucosal tissue at the target location, the outlet may be provided in alternative forms, such as comprising a plurality of injection needles, or as one or more outlet nozzle openings for expelling a substance directly through the outlet by jet action. The disclosed embodiment relates to a capsule 100 suitable for being ingested by a patient to allow the capsule to enter a lumen of the GastroIntestinal tract at a target location, more specifically the small intestines, and configured so that when situated at the target location, the capsule automatically triggers to eject a liquid dose of a drug substance into tissue of the lumen wall surrounding the lumen. In other embodiments, the capsule may be configured for expelling a substance at other locations of the GastroIntestinal system, such as the large intestines, or even at other lumen parts of a subject.

Referring to FIG. 1, the shown cross-sectional view depicts the capsule 100 in a state subsequent to swallowing but prior to activation for drug delivery. Not shown in FIG. 1 is a condition responsive arrangement which prevents activation until a predetermined condition is met, e.g. wherein the condition may be the arrival of the capsule at the target location after swallowing. In the embodiment capsule 100, an enteric coating or other pH sensitive coating (not shown), serves to protect the capsule during its travel through the stomach of the patient but is designed to degrade as the capsule arrives at the target location in the small intestines to enable activation for delivery of drug substance. In accordance with various different embodiments according to the invention, alternatives to an enteric coating may be used instead. As a non-limiting example, a release mechanism may be provided having a dissolvable plug that degrades at a specific location within the GI tract to release a first component relative to second component of the device for relative movement so as to enable or cause activation of delivery of drug substance.

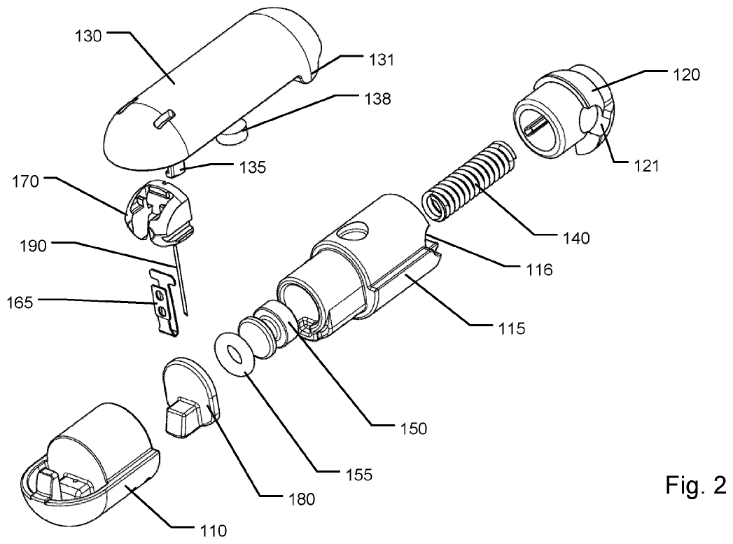

Accompanying FIG. 1, the components of capsule 100 are furthermore depicted in exploded view in FIG. 2. The capsule 100 according to the first embodiment comprises a housing consisting of three housing portions, i.e. a distal (outlet end) housing portion 110, a proximal housing portion 120 and an intermediate housing portion 115. Each of the housing portions are provided as generally tubular shaped members which when assembled relative to each other provides a near oval shaped capsule exterior having a rounded elongated shape which extends along a longitudinal axis. With the three housing portions assembled a generally tubular space extending along the longitudinal axis is provided within the capsule 100 providing a reservoir A for accommodating the drug substance and a delivery assembly configured to force the drug substance through the outlet of capsule 100.

Referring to the reservoir A, inside capsule 100, a hollow cylindrical bore extending along the longitudinal axis of the capsule 100 is formed within intermediate housing portion 115. A distally arranged end wall closes off the cylindrical bore at its distal end, the end wall being provided as a seal member 180 formed from an elastomeric material. A piston 150 is arranged for axial slidable movement inside the hollow cylindrical bore. The piston 150 includes a circumferentially arranged seal 155 that seals against the radially inwards surface of the cylindrical bore. The piston 150 includes a distal facing circular surface arranged for liquid contact and a proximally facing surface arranged to receive a load from an actuator. When the capsule 100 assumes the initial state, i.e. prior to administration, the piston 155 is disposed in a start position remote from distally arranged end wall 181. A liquid drug substance is accommodated in the drug chamber A within cylindrical bore and axially between the piston 150 and the end wall 181.

In the shown embodiment, the actuator is provided in the form of a helical compression spring 140 which has been arranged in a compressed state between a proximal spring seat formed in proximal housing portion 120 and a spring receiving surface arranged on the proximal facing surface of piston 150. Compression spring 140 stores sufficient potential energy to enable the piston 150 to be driven axially to an end position wherein the piston engages end wall 181.

The outlet of capsule 100 comprises an injection needle 190 disposed at the distal end of the capsule. The injection needle 190 is arranged transversely to the longitudinal axis of capsule 100 so that the needle is stored in a lateral orientation inside the housing and will become moved laterally to extend radially outside of the capsule housing through an outlet opening disposed in a side wall of the distal housing portion 110 to enable expelling of the drug substance. In the shown embodiment, when stored inside capsule 100, the injection needle 190 extends along a substantive portion of the width of the capsule and includes a sharpened tip at its front end. A side hole 192 is arranged approximately midways between the rear end and the front end of the injection needle. A lumen extends inside the injection needle 190 from the side hole to a lumen opening at the sharpened tip. The rear portion of the needle is blinded so that liquid drug entering the side hole 192 can only exit at the sharpened tip.

In the shown embodiment, the reservoir A is in fluid communication with a minor appendix forming an outlet pocket 185 arranged adjacent the distal end of reservoir A. The outlet pocket 185 is formed integrally into seal member 180 so that outlet pocket 185 extends distally relative to the distally arranged end wall of reservoir A. The cross-sectional transverse area of outlet pocket 185 compared to the cross-sectional area of the cylindrical bore is small. In the storage state of capsule 100 the front half portion of the injection needle 190 is disposed so that it extends laterally through outlet pocket 185 in a manner wherein the injection needle penetrates two opposing side walls of outlet pocket 185 formed by seal member 180. In the storage state, the side hole 192 and the lumen opening at the sharpened tip are located outside outlet pocket 185 at opposing sides thereof. Hence, the reservoir A and the interior of outlet pocket 185 are sealed when the capsule assumes the storage state even though the injection needle penetrates the side walls of outlet pocket 185. However, by moving the injection needle 190 laterally so that the tip end of the needle extends radially outside the housing of capsule 100, the side hole 192 arrives at the centre of the outlet pocket 185, thereby creating a fluid passage from reservoir A through lumen of injection needle 190 all the way to the lumen opening at the sharpened tip. In this way the injection needle 190 and the outlet pocket 185 forms a delivery gate for controlling delivery from the capsule 100.

Inside the capsule 100 the injection needle 190 is mounted fixedly in a needle carrier 170 which is linearly movable transversely back and forth, i.e. down and up in the figures, between a needle non-exposed position and a needle exposed position. Respective interfacing surface geometries formed in distal housing portion 110 and needle carrier 170 define the transverse movement between the needle carrier and the distal housing portion 110 and define end blocking relative positions between the two, both when the needle carrier assume the needle non-exposed position and the needle exposed position.

In order to activate the capsule 100 to initiate drug delivery, the capsule comprises an activation element 130 that is movable relative to the housing and which is configured to be moved by contractions of the intestinal wall due to peristalsis. The activation element is configured for intestinal wall interaction so that, as a result of muscular contraction in the small intestines, the activation element is moved relative to the housing of the capsule from an expanded pre-de-livery configuration into a non-expanded delivery configuration.

In the shown first embodiment activation element 130 forms an elongated arm that, when the capsule 100 assumes the storage state, lies in intimate relationship along one side section of the capsule housing. In the shown embodiment, cut-outs in the housing of capsule mates with activation element 130 so that the activation element is flush with the outer surface of the housing and wherein the activation element and the housing in combination form an elongated rounded outer surface reminiscent the shape of a size 00 or 000 medical capsule.

The activation element, at the proximal end thereof, includes two opposing bearing surface geometries 131 each of which are configured to be received by cooperating bearing surfaces formed at the proximal end of the housing of capsule 100. Said cooperating bearing surfaces of the housing are formed as pairs of mating bearing surface elements 116/121 formed by intermediate housing portion 115 and proximal housing portion 120, respectively. The bearing surfaces allow the distal end of activation element 130 to rotate away laterally from the distal end of the housing by a predefined angular movement. Hence, the activation element 130 is movable between a non-expanded position and an expanded position, i.e. between the non-expanded pre-delivery configuration and the expanded pre-delivery configuration. The activation element 130 is oriented generally parallel with the longitudinal axis in the non-expanded position and angled with respect to the housing in the expanded position. In the shown embodiment the angular movement of activation element 130 is repeatably performed to control extension of the injection needle 190 from housing, activation of drug delivery through the needle, and subsequent retraction of the injection needle into the capsule.

A biasing element 138 is disposed in a cut-out in intermediate housing portion 115 radially beneath the activation element 130. In the shown embodiment, the biasing element is provided as a biodegradable cellulose sponge that is configured to swell upon being wetted by intestinal juice to thereby expand from the cut-out and transversely force the activation element from the non-expanded position to the expanded position. When being squeezed between the activation element and intermediate housing portion 115, such as during peristatic contractions, the sponge is reduced in radial dimension in accordance with the movement of the activation element 130. Whenever peristaltic contraction ceases the sponge will be able to expand again due to constantly being exposed to intestinal juice and thus to be able to urge the activation element 130 towards its expanded position again.

For the biasing element 138, as an alternative to the sponge, other biasing elements formed separate from the activation element and the housing may be utilized instead, such as by incorporation of compression, tension or leaf spring elements, gas springs or components utilizing gas generation. As an alternative to providing the biasing element as an element formed separately from the activation element and the housing, the biasing element may be formed unitarily with one of the activation element and the housing. In still other embodiments, the activation element and at least one portion of the housing are formed as one unitarily formed element, wherein one or more hinge sections allow the activation element to be moved transversely between expanded and non-expanded positions, such as by a rotational movement or a rectilinear movement relative to the housing portion of the unitarily formed element. In such embodiment, spring elements may be formed by the unitarily formed element so that the activation element is biased towards its expanded position.

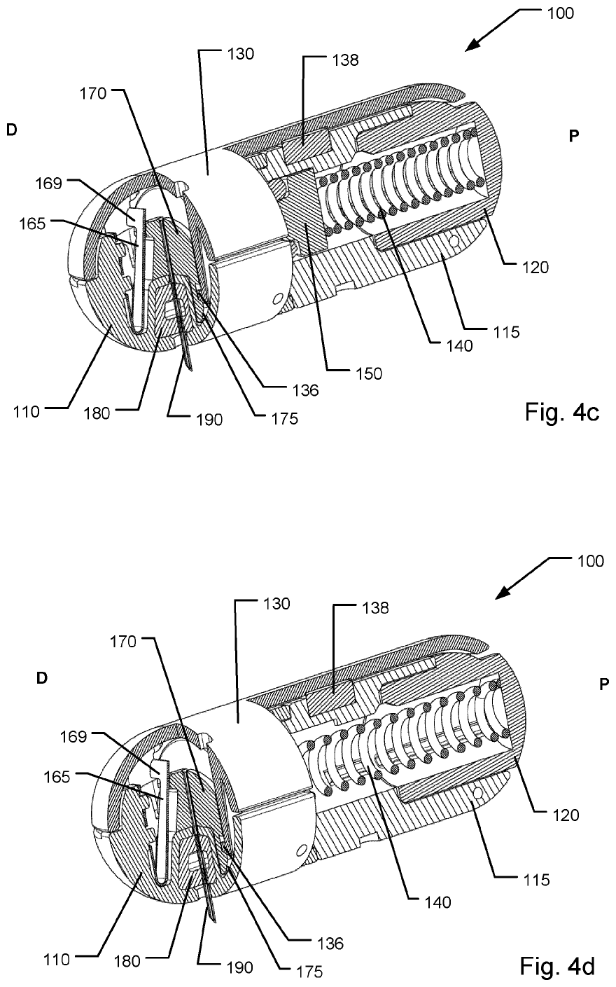

In accordance with the present invention, the activation element of the capsule may be configured to activate delivery from the capsule in a plurality of different ways. In the capsule 100 of the first embodiment, as shown in FIG. 2, and also depicted in FIGS. 4a-4f, the activation element 130 comprises an inward directed snap arm 135 having a snap feature 136 provided at its free end which is configured to cooperate with the needle carrier 170 to control movement of the needle carrier 170 and thus also the transverse position of the injection needle 190. The snap arm 135 is formed so that the free end of the snap arm is radially flexible in a direction transverse to the direction of movement of distal end of activation element 130, i.e. the snap arm 135 is flexible in a radially outward direction relative to the injection needle 190 held by the needle carrier 170.

Needle carrier 170 includes first and second snap receiving geometries 175/177 arranged to receive the snap feature 136 of snap arm 135 at different stages during the operational sequence of the capsule 100. When situated in either of the snap receiving geometries 175/177, the snap feature 136 of snap arm locks temporarily with the respective one of the first and second snap receiving geometries 175/177 to make the needle carrier becoming slaved with the movement of the activation element 130. However, a latch mechanism controls when the needle carrier becomes slaved with the movement of the activation element 130, and when the snap feature 136 of snap arm 135 releases from engagement with the respective one of the first and second snap receiving geometries 175/177 to allow for independent movement of the activation element 130. The sequence of movements will be described later with reference to FIGS. 4a-4f.

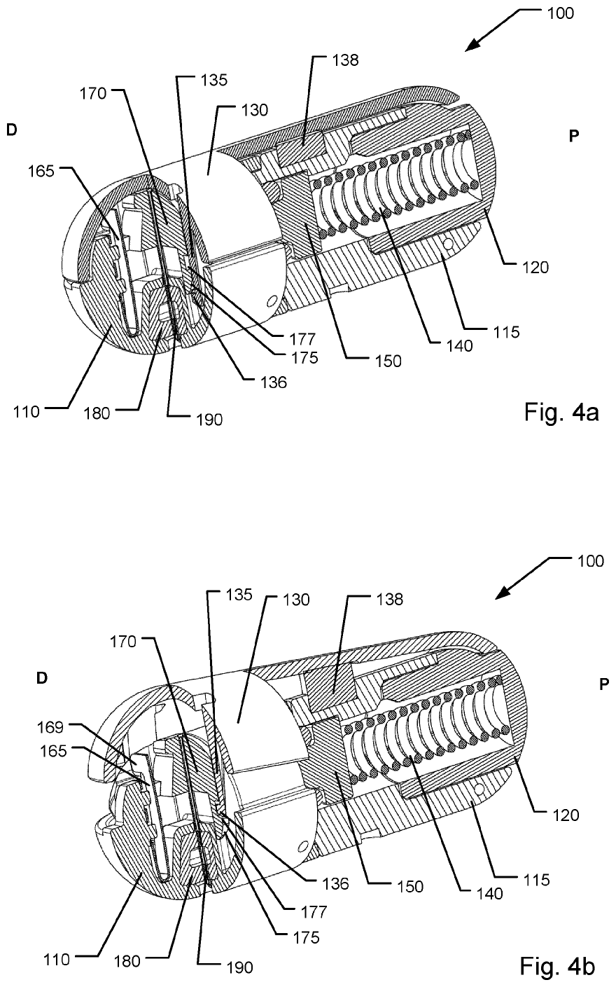

Referring to the three views in FIGS. 3a-3c, the needle hub 170, the injection needle 190 and a latch element 165 are depicted isolated from the remaining components of capsule 100, the views offering a detailed representation of the surface features of the needle carrier 170 and the main details of the latch element 165. In each of the three views the injection needle 190 is seen pointing downwards from the needle carrier 170.

The needle carrier 170 comprises a surface portion 171 facing laterally to both the longitudinal axis of the capsule and laterally to an axis of the injection needle, i.e. meaning that the surface portion points towards the reader when viewing FIG. 1. The surface portion 171 is arranged so that it connects the first and the second snap receiving geometries. The snap feature of snap arm 135 is configured to slide along the surface portion 171 so that, whenever the snap feature rests against surface portion 171, snap arm 135 becomes biased transversely from the injection needle 190 away from a rest position. The first snap receiving geometry 175 and the second snap receiving geometry 177 are both formed as recessed areas relative to the surface portion 171, allowing the snap feature 136 of snap arm 135 to drop into a selected one of the two snap receiving geometries 175/177, thereby temporarily locking the activation element 130 with the needle carrier 170. Due to inclined edge surfaces provided on both the snap receiving geometries 175/177 and the snap feature of snap arm 135 the snap feature of snap arm 135 will be able to become released from the selected snap receiving geometry 175/177 upon increasing force acting to move activation element 130 independently from needle carrier 170. The snap feature will temporarily rest upon surface portion 171 before it drops into the other of the snap receiving geometries.

The latch element 165 is provided as a spring element made from bended flat spring steel so that the latch element 165 is generally U-shaped when arranged in the capsule 100. A first portion of the U-shape 166 is arranged at the distal end of capsule 100 and mounted relative to distal housing portion 110 and the second portion of the U-shape 167 is received within a central slot formed in needle carrier 170. The U-shaped latch element 165 is pre-bended so that the latch element is energized when mounted within capsule 100 with the stored energy urging the second portion 167 axially away from the first portion 166.

The first portion 166 of the U-shape includes two mounting apertures (non-referenced) configured to receive two mounting pins (also non-referenced) extending in the proximal direction from the distal housing portion 110. Due to the mounting pins and mounting apertures the first portion 166 of the latch element is fixedly mounted within the housing of capsule 100. However, with reference to FIGS. 3a and 3b, due to the energy stored in latch element 165, the second portion 167 of the U-shape will be able to move axially from the start position indicated in FIG. 3a and in proximal direction to arrive in the position shown in FIG. 3b, the movement caused and driven by stored energy accumulated in the latch element 165. Apart from this, the needle carrier 170 will be able to move downwards in the needle expelling direction relative to the latch element 165 i.e., from to the needle non-exposed position shown in FIG. 3a, into the needle exposed position shown in FIG. 3b and finally upwards back into the needle non-exposed position shown in FIG. 3c.

The free end of second portion 167 of the latch element 165 defines a T-shaped end section and thus includes two laterally opposed latch portions 169, each extending sideways from the second portion 167, the two latch portions 169 being configured for cooperating with respective blocking elements 179 formed by the needle carrier 170 (compare FIGS. 3a and 3c). A separate latch locking portion 168 is formed by the top of the T-shaped end section of the second portion 167, the latch locking portion 168 being configured to cooperate with a lock protrusion 178 arranged on the needle carrier 170 (compare FIGS. 3a and 3c).

In the first needle non-exposed position of the needle carrier 170 (shown in FIG. 3a) the proximal facing surfaces of the two latch portions 169 rest against corresponding distally facing surfaces of blocking elements 179 formed by needle carrier 170 thereby preventing the second portion 167 against being released for proximal movement.

Upon the needle carrier 170 becoming moved downwards towards the needle exposed position as shown in FIG. 3b, the two latch portions 169 slide off the blocking elements 179 of needle carrier 170, causing latch locking portion 168 of the second portion 167 to be urged proximally to hit the lock protrusion 178 causing the latch locking portion 168 to rest against a top land formed by lock protrusion 178.

As the needle carrier 170 becoming moved back towards the needle non-exposed position as shown in FIG. 3c, the latch locking portion 168 slides off the lock protrusion 178 of needle carrier 170, causing latch locking portion 168 of the second portion 167 to become irreversibly locked beneath a hard edge of the lock protrusion 178. Hence, in this state, the needle carrier 170 is prevented from subsequently moving downwards towards the needle exposed position due to the engagement between the latch locking portion 168 of latch element 165 and the lock protrusion 178 of needle carrier 170.

Next, with reference to FIGS. 4a-4f, the operation of the capsule 100 according to the first embodiment will be described. As discussed previously, the capsule may include an enteric coating that prevents activation of the capsule prior to arrival at the target location in the small intestines. In order to prevent this, the activation element 130 must be prevented from moving from the expanded position towards the non-expanded position.

In the first embodiment, this requirement is met in that the activation element 130 is prevented from moving from the non-expanded position assumed during storage of the capsule 100 to the expanded position by means of the enteric coating. The enteric coating may be arranged to at least cover the activation element prior to dissolution of the enteric coating so as to maintain the activation element in the non-expanded configuration. In other embodiments, the enteric coating may be applied so that it covers the entire outer surface of the capsule. In still other embodiments, the activation element 130 is maintained in the non-expanded position by a covering of the biasing element 138 ensuring that no fluid will be available by the sponge of the biasing element prior to the capsule arriving at the target location. In still other embodiments, the capsule of the first embodiment may be accommodated in an outer capsule that degrades when arriving in the small intestines allowing the capsule to be activated only upon arrival at the target location.

FIG. 4a shows the capsule 100 wherein the activation element 130 of capsule 100 has only just been unconstrained by the enteric coating, this state corresponding to the state shown in FIG. 1. The needle carrier 170 is positioned in the needle non-exposed position whereby the injection needle 190 is held protected inside the capsule 100. Hence, the outlet pocket 185 is penetrated by the injection needle, but in way wherein fluid expelling from reservoir A is prevented.

The piston 150 is arranged in its start position under load from the compression spring 140. The latch element 165 second portion 167 is held in its distal position by the two latch portions 169 being retained by blocking elements 179. Hence, the latch element 165 assumes the state shown in FIG. 3a. The activation element 130 assumes its non-expanded position due to the sponge of biasing element 130 has not yet been wetted by intestinal juice. The snap feature 136 of snap arm 135 is received in snap receiving geometry 175 on the needle carrier 170.

In FIG. 4b the sponge of the biasing element 138 has been wetted by intestinal juice and thus expanded thereby forcing the activation element 130 into its expanded position. The needle carrier 170 assumes the upwards position and cannot move further upward relative to the housing of capsule 100. The snap feature 136 of snap arm 135 has travelled past surface portion 171 of the needle carrier 170 and has now dropped into snap receiving geometry 177 (cf. FIG. 4a). The needle carrier 170 still assumes its needle non-exposed position so that the expelling of drug has not yet been initiated. Movement of the needle carrier 170 from the needle non-exposed position into the needle exposed position is enabled as the second portion 167 of the latch element 165 is situated in the distal position and the latch protrusion 178 is not yet locked relative to latch locking portion 168. The capsule 100 now assumes a triggerable state wherein the next wave of intestinal contractions will cause triggering of the capsule by forcing the activation element 130 back towards its non-expanded position.

In FIG. 4c the activation element 130 has been forced from the expanded position into the non-expanded position due to the contractional forces occurring by peristalsis. The sponge of the biasing element 138 has been squeezed by muscular contractions. The needle carrier 170 has been slaved for movement with the activation element 130, i.e. from the needle non-exposed position into the needle exposed position, due to the engagement between snap feature 136 of snap arm 135 and the snap receiving geometry 177. The front section of the injection needle 192 has been extended radially outwards from the capsule housing and inserted into mucosal tissue of the lumen wall. As the injection needle 190 has been moved into the exposed position the side hole 192 of the injection needle is now positioned centrally into outlet pocket 185 and fluid communication between reservoir A and the lumen opening of the tip of injection needle 190 has been established. In the state shown in FIG. 4c triggering of drug expelling has only just been initiated. The latch element 165 second portion 167 has been released from its distal position as the two latch portions 169 has disengaged from blocking elements 179. The latch element 165 now assumes the state shown in FIG. 3b.

FIG. 4d shows the capsule in the state wherein piston 150 has been moved distally by compression spring 140 into engagement with the distal wall of the reservoir A. Thus, all expellable drug accommodated in reservoir A has been expelled through the injection needle 190 and deposited in the lumen wall at the target site.

FIG. 4e shows the state of the capsule when muscular contractions have ceased, and the sponge of the biasing element 138 has been wetted again by intestinal juice and thus ex-panded, forcing the activation element 130 into its expanded position. The snap feature 136 of snap arm 135 is still engaged by snap receiving geometry 177. Thus, the movement of activation element 130 slaves movement of the needle carrier 170 from the needle exposed position upwards into the needle non-exposed position and the injection needle 190 is accordingly withdrawn from tissue at the target location. Due to the upwards movement of the needle carrier 170 into the needle non-exposed position the latch locking portion 168 of latch element 165 slides off the lock protrusion 178 of needle carrier 170, causing latch locking portion 168 of the second portion 167 to become irreversibly locked beneath a hard edge of the lock protrusion 178 (cf. FIG. 3c). Hence, from this moment, the needle carrier 170 is prevented from moving downwards relative to the housing and has now become safely lodged within the capsule 100.

Finally, FIG. 4f depicts the state wherein a further contractional wave during peristalsis has moved the activation element 130 into its non-expanded position again. As the needle carrier 170 remains locked relative to the housing the further movement of activation element 130 back and forth between the expanded position and the non-expanded position has no influence on other components of the capsule and the capsule 100 will proceed towards excretion. A further not shown mechanism may be included serving to maintain the activation element 130 locked irreversibly into its non-expanded position upon the capsule 100 entering the state shown in FIG. 4f, such as by having the activation element 130 cooperate with latch element 165.

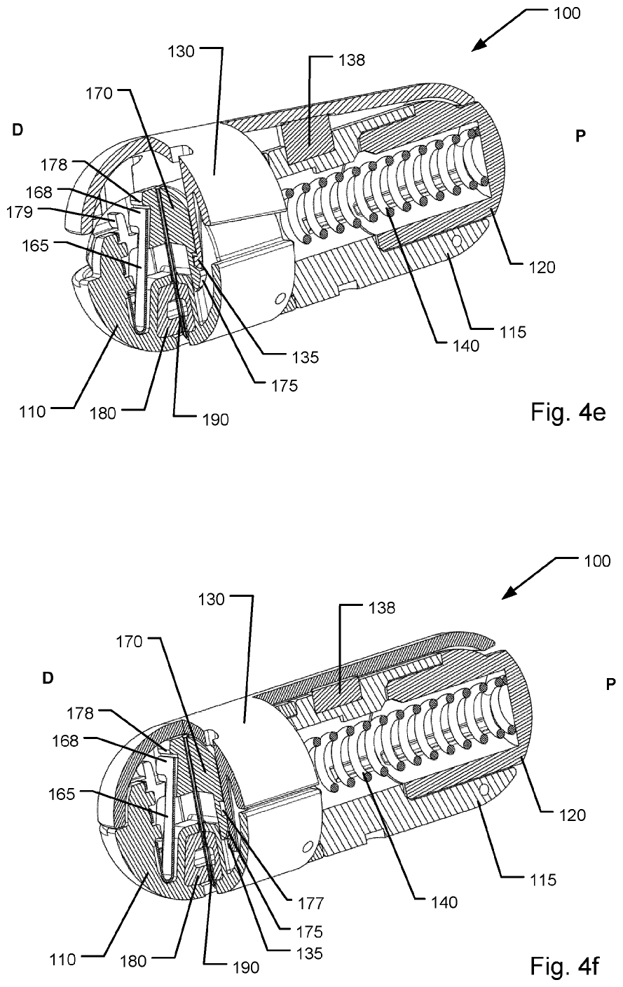

Referring now to FIG. 5a-5c, a second embodiment of a capsule 200 will now be described. The capsule 200 corresponds in many aspects to the capsule 100 but the actuator 140 of the capsule 200 is different. The activation principle and the outlet system of capsule 100 have been reused in the second embodiment capsule 200 and operation thereof fully corresponds to the operation principle of the first embodiment capsule 100. The seal member 280 is formed dif-ferently from seal member 180 but the overall function of seal member is the same for the two embodiments. Also, instead of the housing component comprising three different components, the distal housing portion and the intermediate housing portion have been formed instead as unitary component providing distal housing portion 210 that directly attached to proximal housing portion 220.

Instead of relying on an energized spring serving to store potential energy for providing a load onto the piston of the capsule, the capsule 200 includes an actuator 240 incorporating a gas generator which generates high pressure gas in an actuation chamber B of capsule 200 which in this way serves as an energy source for potential energy. A gas generator capable of producing a gas for driving forward the piston 250. In the shown embodiment, the gas generator is arranged inside a hollow cylindrical section of the capsule which is located proximally from the piston 250 and which forms an actuation chamber B.

Gas may be generated by chemical reaction so that once the gas generator is actuated gas is produced to form pressurized gas in the actuation chamber b of capsule 200. Different principles may be used for providing gas generation inside the actuation chamber B, for example by using a gas producing cell, such as a hydrogen cell, an airbag inflator, a gas generator utilizing phase change, or a generator which incorporates mixing of reactants to chemically react to form a gas, such as by mixing sodium bicarbonate and acid. For gas generation using mixing of reactants, either all reactants may be stored on board the capsule prior to actuation, or at least one reactant may be introduced into the capsule for mixing with a reactant stored on board the capsule.

The following are examples of chemical reactions which produce carbon dioxide CO2 and which may be used as the components for generating pressurized gas in the actuation chamber B:
Example 1 (calcium carbonate with hydrochloric acid): CaCo3+2HCl→CaCl₂)+H2O+CO2
Example 2 (citric acid with sodium bicarbonate): C6H8O7+3NaHCO3→3H2O+CO2+Na3C6H5O7
Example 3 (tartaric acid with sodium bicarbonate): H2C4H4O6+2NaHCO3→Na2C4H4O6+2H2O+2CO2

Examples of acids for effervescent reaction:
Citric acid
Acetic acid
Hydrochloric acid
Tartaric acid
Malic acid
Adipic acid
Ascorbic acid
Fumaric acid

Examples of carbonate salts for effervescent reaction:
Sodium bicarbonate
Sodium carbonate
Calcium carbonate
Potassium bicarbonate

In other embodiments, the effervescent reaction may occur by one or more solid state components being wetted (e.g. exposed to intestinal fluid or other fluid stored within the capsule) which causes the effervescent reaction.

In the embodiment shown in FIGS. 5a-5c, the gas generator of actuator 240 is configured for being manually activated by the user, meaning that the user needs to manually activate the gas generator prior to swallowing the capsule. In order to do this, in the storage state of the capsule 200, an activation rod 246 is incorporated with capsule 200, the activation rod being configured for either being manually gripped by the fingers of a hand, or alternatively being manipulated by means of removal from packaging material (not shown), e.g. upon removal of the capsule from the packaging material, the activation rod is automatically moved for activating the gas generator inside capsule 200.

In the shown second embodiment of capsule 200, gas is generated in the actuation chamber B by means of an internally arranged effervescent material 241 arranged in a distal portion of the actuation chamber B, and an on-board volume of water W in the proximal portion of the actuation chamber B, the two materials being separated by a membrane during storage of the capsule.

Referring to FIG. 5a, which shows the capsule 200 in the storage state, the piston 250 includes a hollow space at the proximal end of the piston, the hollow space accommodating a portion of effervescent material 241. Said membrane, provided as a secondary piston 245 is arranged slideable in the actuation chamber B, and includes a circumferentially arranged sealing lip 248 which seals the proximal end portion from the distal end portion of the secondary piston 245. The secondary piston 245 is in the storage state located at a first distal position and is configured to be moved proximally for mixing the effervescent material and the water. The sealing lip 248 acts as a one-way valve allowing water W from the proximal end of actuation chamber B to pass the secondary piston 245 into the distal portion of the actuation chamber to start the effervescent reaction.

The activation rod 246 is in the shown embodiment made integral with the secondary piston 245 and serves as a pull rod for pulling the secondary piston 245 proximally in the actuation chamber B. Activation rod 246 includes a frangible portion 247 which allows the activation rod 246 to be separated from secondary piston 245 but only subject to the secondary piston 245 has been moved fully proximally in actuation chamber B. Furthermore, the actuation chamber B includes a proximal arranged opening wherein a tubular seal element 249 is arranged. The tubular seal element has an aperture through which the activation rod 246 protrudes so that the tubular seal element 249 effectively seals actuation chamber B from the external environ-ment.

FIG. 5b shows the state of capsule 200 after a user has pulled the activation rod 246 fully proximally. The water W originally stored in the proximal portion of actuation chamber B has bypassed the secondary piston 245 so that the water allows mixing with the effervescent material 241 stored in the distal portion of actuation chamber B. Immediately, gas generation is initiated which acts to build up pressurized gas in the actuation chamber B. However, due to the reservoir A storing the drug substance being still closed in the outlet pocket 285 the piston 250 will only move distally in a non-substantive manner as gas pressure builds up.

FIG. 5c shows schematically the final preparation step of removing the activation rod 246 from the capsule 200 by the user breaking off the activation rod 246 at the frangible portion 247. The capsule is thereafter in a condition suitable for being swallowed by the user. In different embodiments, gas generation in actuation chamber B will either build up pressure before swallowing or will initiate building up pressure to increase pressure during the capsule 200 travelling through the gastrointestinal system of the user. For operation of activation capsule 200 due to peristalsis, reference is made to FIGS. 4a-4f which illustrate a similar activation procedure.

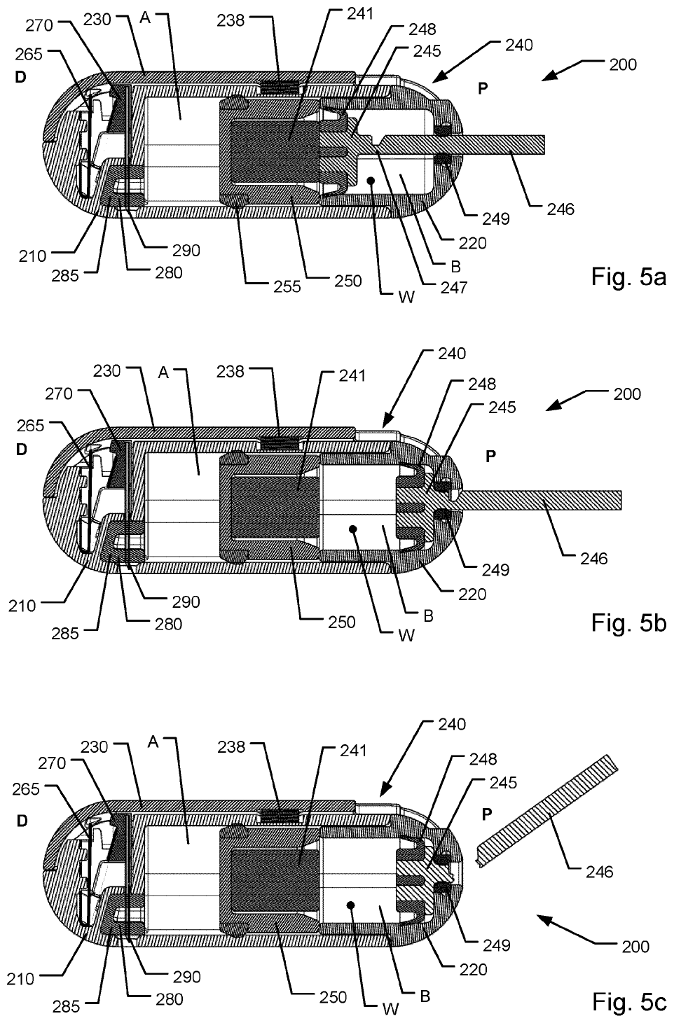

FIG. 6 shows a third embodiment capsule 300 which in many aspects works similar to the second embodiment capsule 200. Instead of using an actuator 240 that requires the user in a handling step to manually initiate the gas generation prior to swallowing, the third embodiment utilizes an actuator 340 wherein gas generation is automatically initiated subsequent to the capsule 300 being swallowed.

Again, the activation principle and the outlet system of capsule 100 have been reused in the third embodiment capsule 300 and operation thereof fully corresponds to the operation principle of the first embodiment capsule 1001n capsule. However, in the third embodiment capsule a water container 342/343 is incorporated as part of the actuation chamber B. The distal portion of actuation chamber B again includes effervescent material 341 which in this embodiment is located proximally to a proximal end face of piston 350. The water W container includes a first major shell portion 342 and a lip portion 343. In the shown embodiment, the shell portion 342 and lid portion 343 are made of resilient material where the interface between the two is sealed prior to or during the mounting process within capsule 300. The lid portion includes a pre-cut slit valve 344 serving as a control valve which is initially maintaining the container in a sealed state, but which allows water W to escape from the water container when pressure inside the water container becomes elevated, subsequently causing the water W to mix with the effervescent material 341.

Proximal housing portion 320 includes a blind bore extending coaxially with the bore in intermediate housing portion 315, i.e. the latter partly defining part the reservoir A. In the proximal end of proximal housing portion 320 a plurality of openings 323 are formed to allow gastric fluid to enter into the proximal end of a blind bore. The water container 342/343 is disposed in a circumferentially sealing manner into said bore so that water W present in the water container can only escape in the distal direction through pre-cut slit valve 344 in the lid portion 343 of the water container. A small volume at the bottom of the blind bore, “a sponge space”, is arranged proximally to water container 342/343 and accommodates a cellulose sponge 345 in a manner which enable fluid exposure with gastric fluid entering through openings 323. Upon exposure to a biological fluid subsequent to swallowing the capsule 300, the sponge 345 will swell and generate pressure within the sponge space causing the water container to be urged in the distal direction. This will deform the water container 342/343 causing the internal fluid pressure within the container to rise and the pre-cut slit valve 344 to open.

In alternative not shown embodiments, to enable the sponge to be quickly wetted, a semi-permeable membrane may be arranged between the openings 323 and the sponge 345 to quickly soak in gastric fluid through openings 323 to provide for transport of liquid into the sponge space. A salt or similar material may be positioned in contact with both the semi-permeable membrane and the sponge portion to serve in combination with the semi-permeable membrane as an osmotic drive.

As soon as water W escapes the water container 342/343 the water begins mixing with the effervescent material 341 stored in the distal portion of actuation chamber B. Immediately, gas generation is initiated which acts to build up pressurized gas in the actuation chamber B. Due to the increased gas pressure pre-cut slit valve will remain open but gas cannot bypass the container portion 342. Due to the reservoir A storing the drug substance being still closed in the outlet pocket 385 the piston 350 will only move distally in a non-substantive manner as gas pressure builds up in the actuation chamber B. As an alternative to the pre-cut slit valve, a burst gate may be arranged between the water W of the actuation chamber B and the effervescent material 341, wherein the burst gate may be configured to irreversibly break open upon elevated liquid pressure in actuation chamber B.

For the operation of activation of the third embodiment capsule 300 due to peristalsis, reference is made to FIGS. 4a-4f which illustrate a corresponding activation procedure.

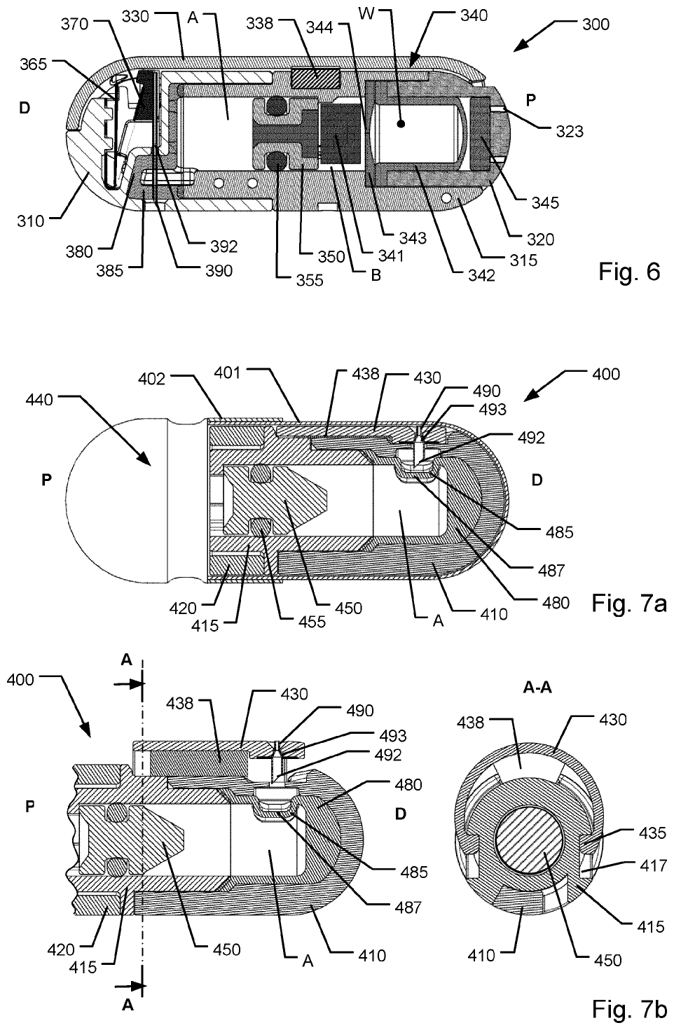

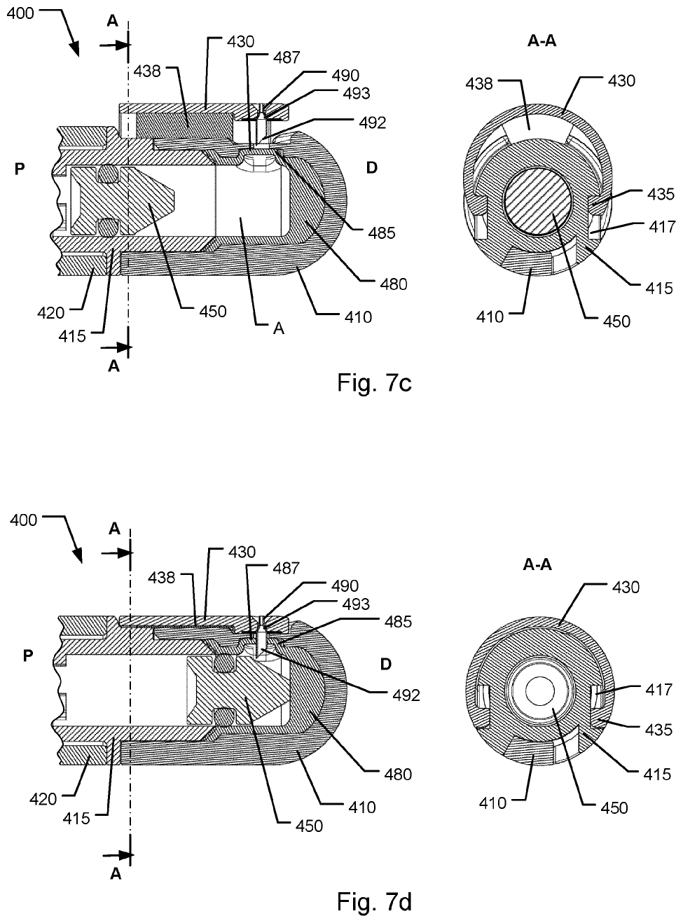

Next, a fourth embodiment capsule 400 will be described, initially referring to FIG. 7a and subsequently to FIGS. 7b-7d. Referring to FIG. 7a, the shown cross-sectional partly cut view depicts the capsule 400 in a state ready to be swallowed.

The ingestible capsule 400 again includes a distal housing portion 410, and intermediate housing portion 415 and a proximal housing portion 420. The functional part of capsule device 400, i.e. all parts needed for the activation and delivery of drug, are initially accommodated in an outer capsule shell 401/402 which in a known manner is configured to degrade after entering the gastrointestinal tract and subject to the outer capsule has been exposed to gastric fluid for a prescribed duration of time.

A slidable piston 450 is again disposed within a longitudinal bore of the housing of the capsule, in the drawing the piston 450 assumes the start position prior to activation of the capsule. The capsule 400 includes an actuator 440 which is only schematically referred to in the drawing shown in FIG. 7a. The actuator 440 includes an energy source providing potential energy which exerts a load onto a proximal face of the piston 450 acting to urge the piston in the distal direction. The energy source stores sufficient potential energy to enable the piston 450 to be driven axially to an end position wherein the piston engages an end wall of substance reservoir A. In certain embodiments of the capsule 400 the actuator 440 may be formed to include a gas generator similar to the gas generator discussed in connection with the third embodiment capsule 300.

The reservoir A comprises a reservoir outlet section 480 which includes a small bi-stable sub-section forming a reversible liquid pocket 485 that selectively can assume two different configurations. In a first non-active configuration the liquid pocket section assumes a shape wherein it reaches radially into the reservoir A, whereas, in a second active configuration which may be referred to as an “inverted configuration”, the liquid pocket section assumes a shape wherein it extends radially outside of reservoir A. A penetrable seal is formed unitarily with the reservoir outlet section 480, the seal being provided as part of liquid pocket 485 i.e., by a wall section 487 oriented with its exterior surface facing radially outwards. The wall section 487 is configured for being penetrated by a piercing element which forms part of the outlet, wherein relative radial movement between the piercing element and the wall section 487 towards each other causes the piercing element to penetrate through the penetrable seal for creating liquid communication across the wall section 487.

In the shown embodiment capsule 400, the outlet is arranged at the distal end of reservoir A. The outlet comprises a jet nozzle 493 dimensioned and shaped to create a liquid jet stream of drug substance when the drug is forced from the reservoir A through the outlet 490. The outlet is formed as a short tubular spike component 490 disposed radially outside reservoir A. A radially inwards pointing spike section 492 of the spike component 490 enables secure penetration of the seal 487. The spike component 490 is arranged in capsule 400 in a way allowing the spike component 490 to be moved radially outwards and inwards between an expanded position and non-expanded position.

In the fourth embodiment, an activation element 430 forms an elongated member that, when the capsule 400 assumes the storage state, lies in intimate relationship along one side section of the capsule housing 410/415/420. In the shown embodiment, cut-outs in the housing of capsule 400 mates with activation element 130 so that the activation element is flush with the outer surface of the housing. The activation element and the housing in combination form an elongated rounded outer surface reminiscent the shape of a size 00 or 000 medical capsule.

The activation element 430 may be coupled to the housing of capsule 400 utilizing one of several different mechanical principles. In the shown embodiment, the activation element 430 is rectilinearly movable relative to housing 410/415/420 by means of linear recessed tracks 417 in distal housing portion 410 which receive coupling guides 435 extending from the activation element 430 in an embracing manner, see FIG. 7b, right view. The tracks 417 and guides 435 enable the activation element to be moved linearly between expanded and non-expanded positions relative to the housing.

Similar to the first embodiment, a biasing element 438 is disposed in a space radially beneath the activation element 430. In the shown embodiment, the biasing element is provided as a biodegradable cellulose sponge that is configured to swell upon being wetted by intestinal juice to thereby expand and transversely force the activation element from the non-expanded position to the expanded position. When being squeezed between the activation element and the housing, such as during peristatic contractions, the sponge is reduced in radial dimension in accordance with the movement of the activation element 130. Whenever peristaltic contraction ceases the sponge will be able to expand again due to constantly being exposed to intestinal juice and thus to be able to urge the activation element 130 towards its expanded position again.

In the fourth embodiment capsule 400, the outlet 490, i.e. the spike component 490, is mounted fixedly relative to the activation element 430 and follows the activation element when moved radially between the expanded position and the non-expanded position relative to the housing. By this arrangement, the radially inwards pointing spike section 492 of the spike component 490 is able to penetrate the penetrable seal at wall section 487 when the activation element 430 assumes the non-expanded position but only when reservoir outlet section 486 assumes the second active configuration, i.e. when the liquid pocket section is extended relative to reservoir A. When the reservoir outlet section 486 assumes the first non-active configuration, i.e. when the liquid pocket section reaches into reservoir A the radially inwards pointing spike section 492 of the spike component 490 does not reach sufficiently far towards the seal at wall section 487 and the seal will remain intact irrespective if the activation element 430 assumes the expanded position and the non-expanded position.

The reservoir outlet section 486 assumes the first non-active configuration whenever the pressure exerted onto piston 450 is below a threshold pressure magnitude. However, when gas pressure from the gas generator of actuator 440 exerts a larger pressure than the threshold pressure magnitude the reservoir outlet section 486 will “flip” into its active configuration.

Next, with reference to FIGS. 7b-7d, the operation of the capsule 400 according to the fourth embodiment will be described. As discussed previously, the capsule is initially accommodated in an outer capsule shell 401/402 which is configured to degrade only after entering the gastrointestinal tract and subject to the outer capsule has been exposed to intestinal juice for a prescribed duration of time. This prevents activation of the capsule prior to arrival at the target location in the small intestines.

In the state shown in FIG. 7b, the outer capsule shell has degraded to a degree wherein the activation element 430 is not retained in the non-expanded position. Hence, intestinal juice has allowed biasing element 438 to swell, causing the expanding sponge to move the activation element 430 from the non-expanded position to the expanded position. The pressure from the gas generator exerts a pressure onto piston 450 below the threshold pressure magnitude. Hence, the reservoir outlet section 486 assumes the first non-active configuration, i.e. wherein the liquid pocket section reaches into reservoir A and no drug expelling is possible.

FIG. 7c shows the capsule 400 when the pressure from the gas generator exerts a pressure onto piston 450 above the threshold pressure magnitude. Hence, the reservoir outlet section 486 has moved into its active configuration. This has been accompanied by a slight distal movement of piston 450. In the shown state, the activation element 430 still assumes the ex-panded position so the radially inwards pointing spike section 492 of the spike component 490 is not able to penetrate the penetrable seal at wall section 487.

FIG. 7d shows the capsule 400 when the activation element 430 has been forced from the expanded position into the non-expanded position due to the contractional forces occurring by peristalsis. The sponge of the biasing element 438 has been squeezed by muscular contractions. The spike component 490 has been moved with the activation element 130 so that the radially inwards pointing spike section 492 of the spike component 490 has penetrated the penetrable seal at wall section 487 and substance delivery by jet action has taken place. Accordingly, the piston 450 has moved distally into the end position and jet delivery has ended.

Should the pressure from the gas generator exert reach a pressure magnitude wherein the pressure onto piston 450 is above the threshold pressure magnitude while the activation element 430 assumes the non-expanded position corresponding to the state shown in FIG. 7a, the delivery by jet action will initiate immediately as soon as the reservoir outlet section 486 “flips” into its active configuration. However, as the activation element 430 in this instance is held in the non-expansive position, this will be due to muscular contractions due to peristalsis pressing on the activation element for moving it to its non-expanded position, and sufficient intimate contact between the intestinal tissue and the outlet has already been assured.

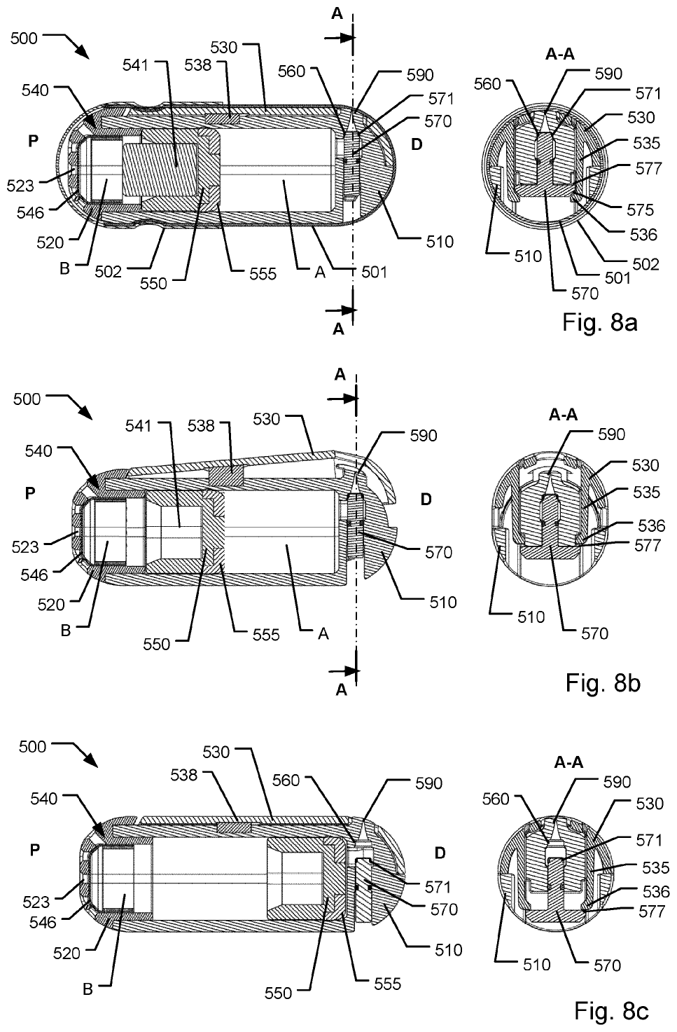

Turning finally to FIGS. 8a through 8c, a fifth embodiment capsule 500 will now be described. Referring to FIG. 8a, the shown cross-sectional view depicts the capsule 500 in a state ready to be swallowed.

The ingestible capsule 500 again includes a distal housing portion 510 and a proximal housing portion 520. The functional part of capsule device 500, i.e. all parts needed for the activation and delivery of drug, are initially accommodated in an outer capsule shell comprising a distal shell portion 501 and a proximal shell portion 502, both of which are formed from degradable material but configured to degrade subject to different conditions in the stomach and the intestinal tract.

A slidable piston 550 is again disposed within a longitudinal bore of the housing of the capsule, in the drawing of FIG. 8a the piston 550 assumes the start position prior to activation of the capsule. The capsule 500 includes an actuator 540 in the form of a gas generator which is based on mixing of reactants wherein one of the reactants include gastric fluid that has mi-grated into a mixing chamber of the capsule 500. An outlet 590 is arranged at the distal end of drug substance reservoir A, the outlet comprising a single jet nozzle configured for delivery of the substance by liquid jet action into the mucosal tissue of the intestinal wall. The jet nozzle is disposed to eject the substance radially away from the capsule at the distal end thereof. In the shown embodiment, the nozzle is formed directly into material portions of the distal housing section 510. A delivery gate function is provided in form of a valve between the reservoir A and the jet nozzle to control delivery from the capsule 500, the delivery gate being controlled by an activation element 530 which is formed in a way resembling the activation element 130 of the first embodiment capsule 100.

Inside capsule 500, at the proximal end thereof, a drive system is arranged configured for driving the piston 550 towards the outlet 590 upon triggering of the drive system, i.e. upon triggering by a predefined condition. The gas generator of the drive system is capable of producing a gas thereby building up gas pressure in actuation chamber B for generating mechanical load onto the piston 550.

The proximal housing portion 520, and more specifically the central planar portion of proximal end wall includes a multitude of openings or channels 523 which allows ingress of gastrointestinal fluid into the actuation chamber B. A semi-permeable membrane 546 is arranged with its proximally facing surface in intimate contact with the distal facing surface of the central planar portion of proximal end wall. Hence, gastrointestinal fluid that enters the capsule 500 needs to pass through the openings 523 and the semi-permeable membrane 546. The central planar portion of proximal end wall provides sufficient rigidity to serve as a backing or support for the semi-permeable membrane 546 when pressure builds up in the actuation chamber B. For the shown embodiment capsule 500 example materials for the semipermeable membrane 546 may be made from Standard Grade Regenerated Cellulose (RC). The material for the semipermeable membrane may be selected so that it is biodegradable when subjected to biological fluid, however only upon being subjected to biological fluid for a period of time considerably longer than the intended time required for delivery of drug substance.

In capsule 500, gas is generated in the actuation chamber B by means of an internally arranged effervescent material 541 arranged in a distal portion of the actuation chamber B, more specifically within a proximally facing bore of piston 550. Effervescent material portion 541 is formed from powder components that are subsequently compressed into block-shape.

Capsule 500 further includes a delivery control member 570 which forms part of the above-mentioned delivery gate or valve. Delivery control member 570 includes a circumferential sealing arrangement 571 that is sealingly received within circular mouth opening 560 of an outlet section of the reservoir A. Delivery control member 570 is linearly movable transversely to the longitudinal axis of capsule device 500 from an initial blocking position into a releasing position (downwards in the figures). Respective interfacing surface geometries formed in distal housing portion 510 and delivery control member 570 define the transverse movement for the delivery control member 570 relative to the distal housing portion 510 and define relative end blocking positions between the two, both when the delivery control member 570 assume the blocking position and the releasing position.

In order to activate the capsule 500 to initiate drug delivery, the capsule comprises an activation element 530 that is movable relative to the housing and which is configured to be moved by contractions of the intestinal wall due to peristalsis. The activation element 530 is configured for intestinal wall interaction so that, as a result of muscular contraction in the small intestines, the activation element 530 is moved relative to the housing of the capsule from an expanded pre-delivery configuration into a non-expanded delivery configuration.

Similar to the first embodiment activation element 530 forms an elongated arm which, when the capsule 500 assumes the storage state, lies in intimate relationship along one side section of the capsule housing. In the shown embodiment, cut-outs in the housing of capsule mates with activation element 530 so that the activation element is flush with the outer surface of the housing and wherein the activation element and the housing in combination form an elongated rounded outer surface reminiscent the shape of a size 00 or 000 medical capsule.

Activation element 530 is mounted relative to the housing of capsule 500 in the same manner as in the first embodiment capsule 100. This allows the distal end of activation element 530 to rotate away laterally from the distal end of the housing by a predefined angular movement. Hence, the activation element 530 is again movable between a non-expanded position and an expanded position, i.e. between the non-expanded pre-delivery configuration and the ex-panded pre-delivery configuration. The activation element 530 is oriented generally parallel with the longitudinal axis in the non-expanded position and angled with respect to the housing in the expanded position. In the shown embodiment the angular movement of activation element 530 controls movement of the delivery control member 570.

Similar to the first embodiment, a biasing element 538 is disposed in a cut-out in distal housing portion 510 radially beneath the activation element 130 and serves to provide a biasing force onto the activation element 530 towards the expanded position.

the activation element 530 comprises a pair of inwardly directed snap arms 535 each having a snap feature 536 provided at its free end which is configured to cooperate with the delivery control member 570 to control movement of the latter and thus delivery of substance from the capsule 500. Each snap arm 535 is formed so that the free end of the snap arm is radially flexible in a direction transverse to the direction of movement of distal end of activation element 530, i.e. the snap arm 535 is flexible in a radially outward direction.

The delivery control member 570 includes pairs of first and second snap receiving geometries 575/577 arranged to receive the snap feature 536 of the respective snap arm 535 at different times during the operational sequence of the capsule 500. When situated in either of the snap receiving geometries, the snap feature 536 of each snap arm locks temporarily with the respective one of the first and second snap receiving geometries 575/577. When the snap feature 536 of the respective snap arm 535 is situated in the second snap receiving geometry 577, this causes the delivery control member 570 to become slaved with the movement of the activation element 130. The sequence of movements will be described later with reference to FIGS. 8a-8c.

The distal shell portion 501 and the proximal shell portion 502 as depicted in FIG. 8a may be formed to degrade upon being exposed to different conditions. The proximal shell portion 502 may be configured to start degrading already when the capsule 500 is positioned in the stomach, i.e. soon after swallowing. This leaves time for the gas generator 540 of the capsule to build-up sufficient gas pressure in the actuation chamber for a jet delivery action to be enabled when the capsule 500 later arrives in the small intestines. The distal shell portion 501 may be configured to degrade only upon arriving in the small intestines. As taught in the art, an enteric coating may be used in association with the capsule to initiate an action. Hence, distal shell portion 501 may be configured to only release activation element 530 from the non-expanded position upon the enteric coating becomes dissolved in the small intestines, i.e. shortly after arriving in the small intestines, which will cause the activation element 530 to enter the ex-panded position soon after release of activation element from the non-expanded position.

FIG. 8a shows the capsule 500 in a state where it is ready to be swallowed by a user. Both the distal shell portion 501 and the proximal shell portion 502 are intact. The delivery control member 570 assumes the blocking position where fluid communication between reservoir A and jet nozzle of outlet 590 is not yet established. The activation element 530 is in the non-expanded position and the sponge material 538 is correspondingly thin in the radial dimension. The snap feature of the respective snap arm 535 is situated in the first snap receiving geometry 575.

FIG. 8b shows capsule after it has arrived at the target location in the small intestines. After being exposed to gastric fluid in the stomach, the proximal shell portion 502 has degraded and gas generation has initiated due to the osmotic drive and the resulting reaction between the gastric fluid and the effervescent material 541 inside actuation chamber B, thereby providing sufficient gas pressure for the subsequent jet delivery to take place. Hence, potential energy is stored by actuator 540 and mechanical load is exerted onto piston 550. Upon arrival in the small intestines, the distal shell portion 501 has degraded. Hence, activation arm 530 is no longer retained in the non-expanded position and intestinal juice has become available for the biasing element 538 which has expanded and in turn forced the activation element 530 into its expanded position. The snap feature 536 of the respective snap arm 535 is now situated in the second snap receiving geometry 577 (see right view in FIG. 8b). The delivery control member 570 still assumes the blocking position. The piston 550 is still located at the start position or have shifted slightly distally due to the pressure exerted by actuator 540.

In FIG. 8c the capsule 500 has been subject to a first contraction in the intestines due to peristalsis for the first time after assuming the state shown in FIG. 8b. The force of the contractions has moved the activation element 530 to its non-expanded position. As the snap feature of the respective snap arm 535 is now situated in the second snap receiving geometry 577 the delivery control member 570 has been moved due to being slaved by the activation element 530, and the delivery control member now assumes the released position. This has established fluid communication between the reservoir A and the jet nozzle 593 at the outlet 590 and delivery has taken place by jet action into the mucosal tissue of the GI wall. The piston 550 thus has arrived at the end position. Thus, all expellable drug accommodated in reservoir A has been expelled through the jet nozzle and deposited in the lumen wall at the target site. The used capsule 500 are now in a state where it is allowed to pass the alimentary canal and be subsequently excreted.

In the above fifth embodiment, a condition responsive retainer has been provided in the form of a dissolvable distal shell portion 501 which prevents and enables operation of the activation element to control initialisation of drug delivery, e.g. by means of controlling under which conditions the delivery gate is allowed to shift from the closed configuration to the open configuration. However, in other embodiments according to the invention, other types of condition responsive arrangements may be provided which control when the delivery gate shifts from the closed configuration to the open configuration. Non-limiting examples of condition responsive arrangements include latch arrangements which are configured for controlling movement of the activation element from the non-expanded pre-delivery configuration to the expanded pre-delivery configuration. Alternatively, latch arrangements may be provided configured for controlling movement of the activation element, i.e. under which conditions the movement of the activation element from the expanded pre-delivery configuration to the non-expanded delivery configuration becomes enabled.

Still other latch arrangements according to the invention may be provided which are configured to control the mechanical transmission between the activation element and the delivery gate, e.g. a latch configuration which initially prevents the mechanical transmission from transmitting an activation force from the activation element onto the delivery gate thereby preventing initialisation of drug delivery, and which, upon a change in a pre-defined condition, enables the mechanical transmission to transmit an activation force from the activation element onto the delivery gate for initializing drug delivery.

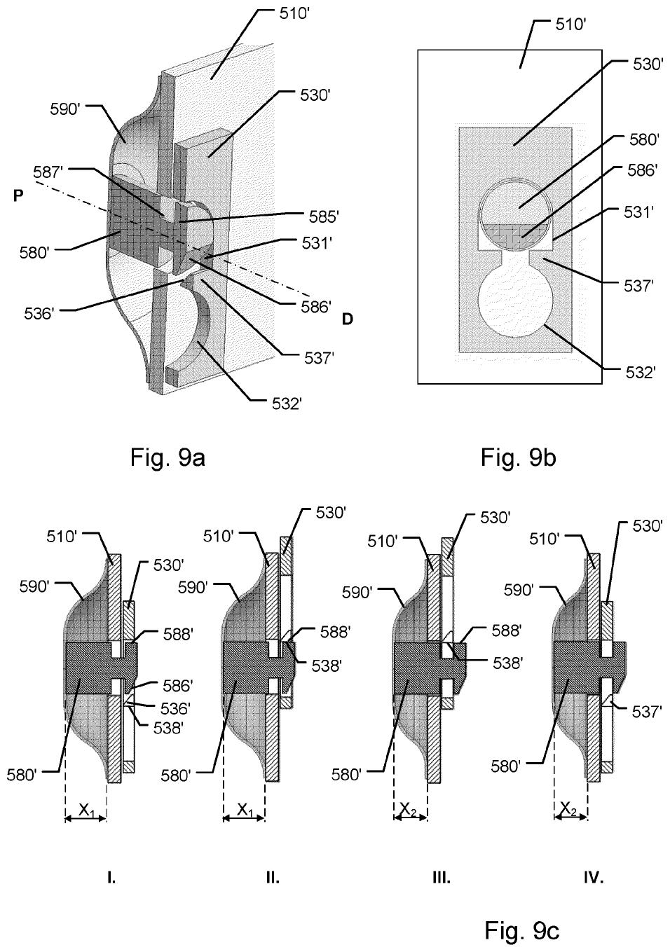

In FIGS. 9a to 9c schematic representations are provided of an example latch arrangement suitable for being included in a modified capsule device (not shown) which may be slightly modified relative to the fifth embodiment capsule device 500 described above. In such modified design, the dissolvable distal shell portion 501 may be omitted. In the latch arrangement shown in FIGS. 9a-9c a pressure operated latch arrangement is shown which may control movement of the activation element from the expanded pre-delivery configuration to the non-expanded delivery configuration but only as a result of sufficient gas pressure being available in actuation chamber B of the capsule device. This is to avoid premature initialisation of drug delivery prior to the required gas pressure level becomes available.

In the modified capsule device, the distal end portion of the capsule device has been modified, i.e. the reservoir outlet portion and the arrangement surrounding the delivery control member 570. The shown latch arrangement is built upon a first housing component distal end wall 510′ which may be provided as a part of distal housing portion 510. A portion of an activation element latch member 530′ is visible in FIG. 9a, this being provided as a part of an activation element being similar in function to activation element 530 of the fifth embodiment. The activation element latch member 530′ is movable laterally to the longitudinally axis of the capsule device and relative to distal end wall 510′ i.e., upwards and downwards in the figures.

A bi-stable flexible membrane 590′ is arranged within drug reservoir A at the distal end thereof, the bi-stable membrane having a circumferential lip arranged in fluid tight engagement with the distal end wall 510′. A central portion of bi-stable membrane 590′ assumes a position with spaced axial relationship X1 with respect to a proximal surface of the distal end wall 510′. Upon increasing liquid pressure within reservoir A the central portion of bi-stable membrane 590′ becomes gradually urged towards the first housing component 510′. When the liquid pressure level within reservoir A exceeds a predefined magnitude the central portion of bi-stable membrane 590′ will move rapidly into a second position in spaced axial relationship X2 smaller than xi.

A central latch pin 580′ is fixedly arranged relative to central portion of bi-stable membrane 590′ in a manner so that it extends from the membrane in distal direction through a hole in distal housing portion 510′ and further through a first opening 531′ of activation element latch member 530′. The central latch pin 580′ follows axial movement of the central portion of bi-stable membrane 590′. Central latch pin 580′ includes a circumferential groove 587′ and a free distal end section that forms an enlarged head 585′ relative to reduced diameter groove 587′.

The groove 587′ has a width in axial direction slightly larger than the axial thickness of activation element latch member 530′. Enlarged head 585′ includes a chamfered lower region on its distal end face and provides an angled surface 586′ (oriented downwards/distally in the FIG. 9a).

Activation element latch member 530′ includes a second opening 532′ adjacent the first opening 531′ and located laterally downwards thereto. Both the first opening 531′ and the second opening 532′ form apertures slightly larger than the enlarged head 585′ of central latch pin 580′. A further narrow cut-out region connects the first opening 531′ with the second opening 532′. The narrow cut-out region is defined sideway by narrow shoulder sections 537′ on each side of the cut-out region. The narrow cut-out region is dimensioned to allow the activation element latch member 530′ to move laterally upwards thereby shifting position so that the central latch pin 580′ is moved from the first opening 531′ to the second opening 532′, however only when central latch pin 580′ is arranged axially so that the reduced diameter groove 587′ is aligned with the activation element latch member 530′. A laterally upwards facing surface portion of shoulder sections 537′ includes a chamfered upper surface and provides an angled surface 536′ (oriented upwards/proximally in FIG. 9a) and thus able to slidingly engage the angled surface 586′ of central latch pin 580′.

Referring to FIG. 9c, views I through IV, a sequence of operating activation latch member 530′ will next be described. In view I the liquid pressure in reservoir A is low due to gas pressure of gas generating actuator having not generated sufficient gas pressure for a satisfactorily drug delivery to take place. Hence, the central portion of bi-stable membrane 590′ assumes a position in spaced axial relationship X1 with respect to a proximal surface of the distal end wall 510′. In this position, the enlarged head 585′ of central latch pin 580′ is axially aligned with the activation element latch member 530′, i.e. in axially overlapping relationship. In view I, the activation element of the capsule device assumes its non-expanded pre-delivery configuration and the enlarged head 585′ is positioned in the first opening 531′.

Upon arrival of the capsule device in the small intestines, the distal shell portion 501 has degraded. Hence, the activation arm is no longer retained in the non-expanded position and intestinal juice has become available for the biasing element 538 to urge activation element 530/into its expanded position, i.e. also urging activation element latch member 530′ upwards. Due to the angled surface 536′ of activation element latch member 530′ and angled surface 586′ of central latch pin 580′, the two angled surfaces are able to slide relative to each other, causing the central latch pin 580′ to move slightly proximally as the activation element latch member 530′ is forced laterally upwards, this being allowed by the resilient nature of bi-stable flexible membrane 590′. The central latch pin 580′ next is caused to snap back distally arriving in the initial axial position. This is depicted in view II. Hence, the activation element 530 is able to move into its expanded pre-delivery configuration although the actuator 540 is not yet in a condition suitable for expelling.

In FIG. 9c, view II it is readily apparent that, with the low liquid pressure within reservoir A, the activation element latch member 530′ cannot be moved laterally downwards relative to distal end wall 510′ due to the surfaces 538′ and 588′, i.e., a downwards facing blocking surface 538′ defined by activation element latch member 530′ and an upwards facing blocking surface 588′ defined by central latch pin 580′.

As depicted in FIG. 9c, view III, only upon sufficient liquid pressure having been formed in reservoir A, corresponding to a predefined target gas pressure level in actuating chamber B, the central portion of bi-stable membrane 590′ assumes a second position in reduced spaced axial relationship X2 with respect to a proximal surface of the distal end wall 510′. In this position, the central latch pin 580′ has arrived axially in a position in a manner wherein the reduced diameter groove 587′ is axially aligned with the activation element latch member 530′. In this position the activation element latch member 530′ is able to slide downwards relative to distal end wall 510′.

Upon the next occurring contraction of the intestinal wall caused by intestinal wall peristalsis the activation element 530/activation element latch member 530′ is forced laterally downwards whereby the shoulder sections 537′ are allowed to pass within the reduced diameter groove 587′ of central latch pin 580′. This is depicted in FIG. 9c, view IV. Hence, as the predefined target gas pressure level in actuating chamber B has been reached, the activation element 530 arm is allowed to move to its non-expanded delivery configuration whereby the delivery gate is forced to initialize drug delivery for a proper dose formation to occur by liquid jet injection into the mucosal tissue of the intestinal wall. Hence, the described pressure operated latch arrangement enables a simple solution for ensuring that the stored potential energy is reached within the actuator of the capsule device for a proper deposition to occur which is optimally synchronized with the peristaltic movements within the small intestine of the intestinal tract.

In the above example the pressure operated latch arrangement is associated with the liquid pressure inside the reservoir A. Instead, a similar pressure operated latch arrangement may be incorporated in a manner wherein it is associated with the gas generator in the actuation chamber B. In still other examples, the pressure operated latch arrangement may be designed to control the movement of the delivery gate so that a movable part of the delivery gate is only caused to open the delivery gate upon the gas pressure level having reached the desired target pressure level.

In still other embodiments, instead of providing the latch so as to prevent the activation element from moving from the expanded pre-delivery configuration to the non-expanded delivery configuration, in alternative embodiments, the pressure operated latch arrangement may be configured for preventing movement of the activation element from the non-expanded pre-delivery configuration to the expanded pre-delivery configuration until the gas pressure level of the actuator having reached the desired target gas pressure level.

In still other exemplary configurations, latch arrangements may be incorporated which relies on parameters other than the gas pressure level or liquid pressure level. For example, a time operated latch system may be incorporated wherein the time operated latch controls the operation of the delivery gate by means of movement of the activation element, but only subject to a predefined time has lapsed, the predefined time being correlated to the time needed for obtaining the desired target pressure level within the actuation chamber.

In the above description of exemplary embodiments and examples, the different structures and means providing the described functionality for the different components have been described to a degree to which the concept of the present invention will be apparent to the skilled reader. The detailed construction and specification for the different components are considered the object of a normal design procedure performed by the skilled person along the lines set out in the present specification.

1. A capsule device suitable for swallowing into a lumen of a gastrointestinal tract of a patient and for intestinal delivery of a therapeutic substance from the capsule device, wherein the capsule device comprises:
a housing portion
an activation element movable relative to the housing portion from an expanded pre-delivery configuration into a non-expanded delivery configuration, the activation element being configured for intestinal wall interaction so that the activation element is moved, due to intestinal wall peristaltic contraction, from the expanded pre-delivery configuration into the non-expanded delivery configuration,
a therapeutic substance comprised in a reservoir (A), and
a delivery assembly comprising a delivery outlet, wherein the delivery assembly is configured for deployment responsive to operation of the activation element to deliver the therapeutic substance through the delivery outlet,
wherein the delivery assembly comprises an actuator comprising a potential energy source, wherein the actuator is configured for driving delivery of the therapeutic substance through the delivery outlet upon release of energy from the potential energy source, and wherein the activation element is configured to initiate delivery of the therapeutic substance as the activation element moves from the expanded pre-delivery configuration into the non-expanded delivery configuration.

2. The capsule device as in claim 1, wherein the reservoir (A) accommodates an amount of the therapeutic substance and wherein, in use, and prior to the activation element moves from the expanded pre-delivery configuration into the non-expanded delivery configuration, the potential energy source either holds an amount of stored potential energy sufficient to deliver all, or holds an amount of stored potential energy sufficient to deliver at least 50 percent.

3. The capsule device as in any of claim 1, wherein a delivery gate is arranged to control transport of the therapeutic substance from the reservoir (A) through the delivery outlet, wherein the activation element is operably connected to the delivery gate for initializing delivery upon movement of the activation element from the expanded pre-delivery configuration to the non-expanded delivery configuration.

4. The capsule device as in claim 3, wherein the activation element mechanically connects to the delivery gate by structure of a mechanical transmission so that, in use, a peristaltic contraction force is transmitted via the activation element and via the mechanical transmission onto the delivery gate to mechanically open the delivery gate to initialize delivery through the drug outlet.

5. The capsule device as in claim 3, wherein the delivery gate defines a first gate element and a second gate element associated with the housing portion and the activation element, respectively, and wherein the first gate element and the second gate element define cooperating elements of an openable seal or openable valve associated with the delivery outlet.

6. The capsule device as in claim 3, wherein the actuator comprises an actuation chamber (B) and wherein the potential energy source comprises a gas generating expansion unit configured for generating gas at elevated gas pressure in the actuation chamber (B).

7. The capsule device as in claim 6, wherein the capsule device further comprises a pressure operated latch arrangement for controlling operation of the delivery gate, the pressure operated latch arrangement configured to release only upon elevated gas pressure being generated above a predefined level in the actuation chamber (B), and/or elevated liquid pressure being generated above a predefined level in the reservoir (A), to enable operation of the delivery gate for initializing delivery.

8. The capsule device as in claim 1, wherein the potential energy source of the actuator comprises one or more of a pre-loaded spring, a compressed gas storage unit, and a gas generating expansion unit.

9. The capsule device as in claim 1, wherein the activation element is releasably maintained in a non-expanded pre-delivery configuration and configured for being released for movement relative to the housing portion from the non-expanded pre-delivery configuration to the expanded pre-delivery configuration.

10. The capsule device as in claim 9, wherein the activation element is releasably maintained in the non-expanded pre-delivery configuration by a condition responsive retainer, such as a dissolvable retainer configured to dissolve when subjected to a biological fluid for release of the activation element.

11. The capsule device as in claim 9, wherein an activation element biasing unit is configured to move the activation element from the non-expanded pre-delivery configuration to the expanded pre-delivery configuration, and wherein the activation element biasing unit comprises one or more of a swelling material portion, a spring, a compressed gas, and a gas generator.

12. The capsule device as in claim 1, wherein the capsule device is configured to act by one of jet delivery of a liquid formulation, jet delivery of a powder formulation, needle delivery of a liquid formulation, and solid dose delivery of a solid dose formulation.

13. The capsule device as in claim 1, wherein the capsule device comprises an injection needle associated with the delivery outlet, wherein the injection needle is configured for movement between a non-exposed state and an exposed state for penetrating intestinal tissue by the injection needle upon movement of the activation element from the expanded pre-delivery configuration into the non-expanded delivery configuration.

14. The capsule device as in claim 13, wherein the activation element is further operable from the non-expanded delivery configuration into an expanded post-delivery configuration, wherein the injection needle moves from the exposed state to the non-exposed state upon movement of the activation element from the non-expanded de-livery configuration into the expanded post-delivery configuration.

15. The capsule device as in claim 1, wherein the capsule device defines an elongated object that extends along a longitudinal axis, and wherein-the activation element forms an element that is radially movable by swivelling or linear movement relative to the housing portion.

16. The capsule device as in claim 2, wherein the amount of stored potential energy is sufficient to deliver at least 60 percent of the amount of therapeutic substance through the delivery outlet.

17. The capsule device as in claim 2, wherein the amount of stored potential energy is sufficient to deliver at least 70 percent of the amount of therapeutic substance through the delivery outlet.

18. The capsule device as in claim 2, wherein the amount of stored potential energy is sufficient to deliver at least 80 percent of the amount of therapeutic substance through the delivery outlet.

19. The capsule device as in claim 2, wherein the amount of stored potential energy is sufficient to deliver at least 90 percent of the amount of therapeutic substance through the delivery outlet.

20. The capsule device as in claim 2, wherein the amount of stored potential energy is sufficient to deliver at least 95 percent of the amount of therapeutic substance through the delivery outlet.
